(19) United States
(12) Patent Application Publication
(10) Pub. No.: US 2015/0157254 A1
(43) Pub. Date: Jun. 11, 2015

(54) METHOD FOR DIAGNOSING SKIN DISEASE BASED ON IN VIVO SKIN IMAGING

(71) Applicant: NATIONAL TAIWAN UNIVERSITY, TAIPEI (TW)

(72) Inventors: CHI-KUANG SUN, New Taipei (TW); YI-HUA LIAO, New Taipei (TW); MING-RUNG TSAI, New Taipei (TW)

(73) Assignee: NATIONAL TAIWAN UNIVERSITY, Taipei (TW)

(21) Appl. No.: 14/164,144

(22) Filed: Jan. 25, 2014

Related U.S. Application Data
(63) Continuation-in-part of application No. 14/098,550, filed on Dec. 6, 2013.

(51) Int. Cl. A61B 5/00

(52) U.S. Cl. CPC A61B 5/444; A61B 5/0064; A61B 5/7282; A61B 2576/02

(57) ABSTRACT

A method for examining skin tissue of a subject based upon in vivo skin imaging includes a harmonic generation microscopy (HGM) device. The HGM device is configured for observing the skin tissue and forming sectioned images of the skin tissue of second harmonic generation (SHG), third harmonic generation (THG), or combination of the SHG and the THG of an excitation light scanning the skin tissue. The method further includes a step to determine whether the morphological feature of the skin tissue is related to a skin disease.

This application is a continuation-in-part of co-pending U.S. patent application Ser. No. 14/098,550, filed on Dec. 6, 2013, and claims priority thereto. The disclosure of the '550 patent application is essentially incorporated herein by reference.

### BACKGROUND

1. Technical Field

The present disclosure relates to technologies of human skin disease diagnosis and particularly to a system and method for examining skin tissue of a subject based on in vivo skin imaging thereof.

2. Description of Related Art

Non-invasive optic imaging techniques for examining and analyzing the microscopic features of tissue is desirable for skin tests. At present, to estimate human chronological age, bone or dental X-ray imaging methods are employed to obtain human bone or dental age which in turn is used to indicate the human chronological age. However, the X-ray imaging methods are harmful due to the exposure to the radioactive X-ray. To solve the problem, the co-pending '550 patent application proposes to use an in vivo skin imaging to estimate the human chronological age. Moreover, to diagnose a skin disease, e.g. skin cancer, biopsy is currently the most accurate method for determining whether a patient has the skin disease. Worse than the X-ray imaging methods, biopsy is an invasive and painful procedure and often causes emotional distresses on patients.

U.S. Pat. No. 6,922,279 which has a same co-inventor with the present disclosure and assigned to the same assignee as the present disclosure discloses a harmonic generation microscopy for providing an image of a biological sample in vivo. No further disclosure in the '279 patent discusses the use of the microscopy to determine human age based on in vivo skin imaging. US Patent Application Publication No. 2013/0001408 A1 which has two same co-inventors with the present disclosure and assigned to the same assignee as the present disclosure discloses a further improvement over the '279 patent. However, the '408 patent publication still does not disclose the use of the microscopic system as a device for determining human age based on in vivo skin imaging.

Additionally, both of the '279 patent and the '408 patent publication neither disclose the use of the microscopic system for examining skin tissue of a subject based on in vivo skin imaging, nor disclose that for diagnosing a skin disease in a subject based on in vivo skin imaging.

Therefore, it is desirable to provide a system and method to examine skin tissue of a subject based on in vivo skin imaging that can overcome the above-mentioned problems.

### BRIEF DESCRIPTION OF THE DRAWINGS

Many aspects of the present disclosure can be better understood with reference to the following drawings. The components in the drawings are not necessarily drawn to scale, the emphasis instead being placed upon clearly illustrating the principles of the present disclosure.

FIG. 1 is a schematic view of a system for estimating human chronological age based on in vivo skin imaging, according to an embodiment.

FIG. 2 is a schematic view showing how the system of FIG. 1 is used to perform a scanning operation on human skin.

FIG. 3 schematically shows exemplary sectioned images of the human skin.

FIG. 4 schematically shows layers of the human skin.

FIG. 5 schematically shows layers of the epidermis of the human skin.

FIG. 6 schematically shows an isolated papillae volume obtained by an analysis device of the system of FIG. 1 for an analysis of the skin in vivo.

FIG. 7 shows graphs of statistical results indicating a relationship between a height of the papilla and the human chronological age.

FIG. 8 shows graphs of statistical results indicating a relationship between a volume of the papilla and the human chronological age.

FIG. 9 shows graphs of statistical results indicating a relationship between a thickness of the epidermis including the papillae and the human chronological age.

FIG. 10 schematically shows a diagram of a junction between the epidermis and the denims obtained by the analysis device of FIG. 1 for another analysis of the skin in vivo.

FIG. 11 shows graphs of statistical results indicating a relationship between a 3D interdigitation index and the human chronological age.

FIG. 12 shows schematic sectioned images of the stratum basale of different subjects.

FIG. 13 shows graphs of statistical results indicating a relationship between a cellular area of the stratum basale and the human chronological age.

FIG. 14 shows graphs of statistical results indicating a relationship between a nucleus area of the stratum basale and the human chronological age.

FIG. 15 shows a series of in vivo HGM images of skin tissue of a patient suffered from malignant melanoma.

FIG. 16 shows a series of in vivo HGM images of skin tissue of a patient suffered from basal cell carcinoma.

FIG. 17 shows a series of in vivo HGM images of skin tissue of a patient suffered from melanocytic nevus.

FIG. 18 shows a series of in vivo HGM images of skin tissue of a patient suffered from seborrheic keratosis.

### DETAILED DESCRIPTION

Embodiments of the present disclosure will be described with reference to the drawings.

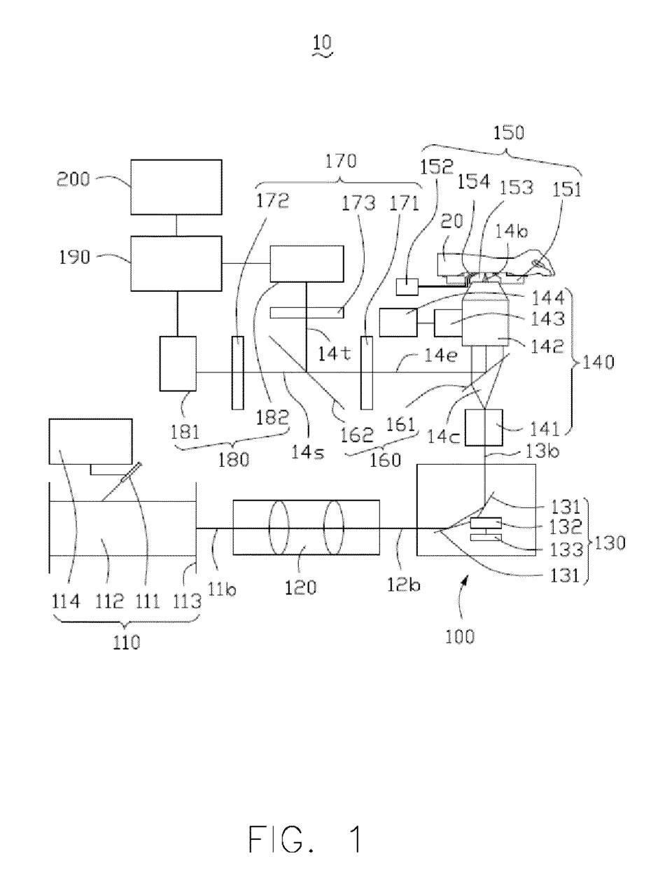

Referring to FIG. 1, a system 10 for human age estimation based on in vivo skin imaging, according to an embodiment, is shown.

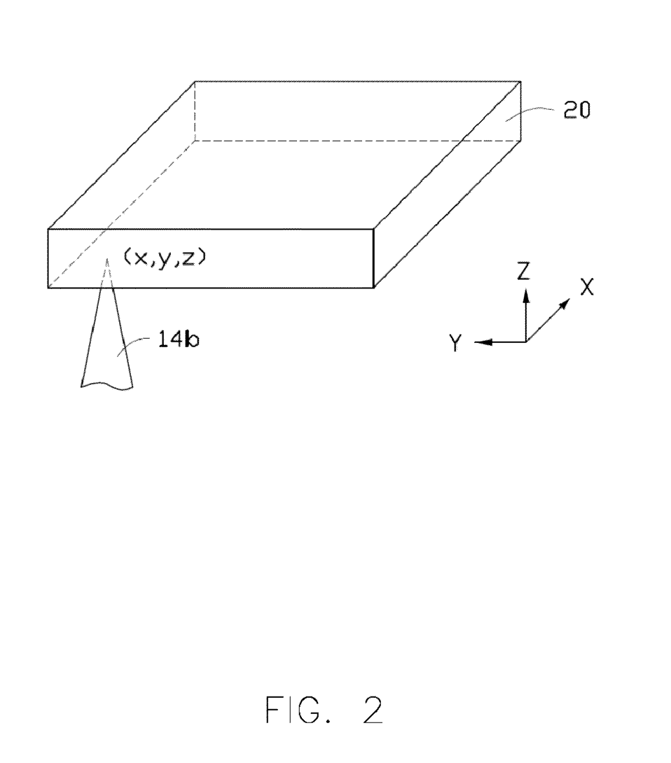

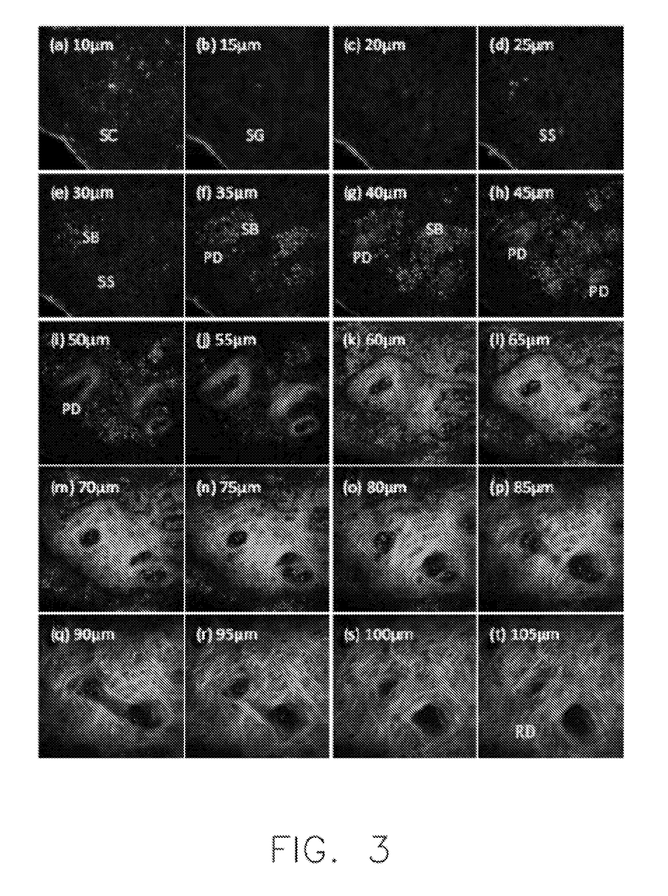

The system 10 includes a harmonic generation microscopy (HGM) device 100 configured for observing human tissues such as skin of leg, forearm, abdomen, and any other suitable portion, whose aging is caused by intrinsic factor, i.e., chronological age. The HGM device 100 is also configured for forming sectioned images of the human skin (see FIG. 3) of harmonic generations, such as second harmonic generation (SHG) and third harmonic generation (THG), of an excitation light that scans the human skin (see FIG. 2). In this embodiment, the SHG and THG are used. However, in other embodiment, a fourth harmonic generation can be used.

The system 10 also includes an analysis device 200. The analysis device 200 is in communication with the HGM device 100 and configured for processing the sectioned images and thus revealing relationships between morphological features of the human skin and human chronological ages.

The relationships can be stored as a database. The analysis device 200 can also be configured for processing the sectioned images of a sample 20 of a person (e.g., the human skin of the ventral forearm about 15 centimeters (cm) of the wrist of the person) to obtain the morphological features of the sample 20 and thus to assist to estimate the chronological age of the person using the database, i.e., the revealed relationships.

In principle, skin aging is a process of structural integrity loss and physiological changes caused by both intrinsic and extrinsic factors. Intrinsic aging of skin is a natural process genetically determined and inalterable. Morphologic changes related to intrinsic aging in older skin are relatively subtle and consist of primary laxity, fine wrinkling, and a variety of benign neoplasms. The changes can be observed and investigated in cellular level and used to estimate the human chronological age.

It is noteworthy that results of the present in vivo skin imaging are also useful for evaluations of skin health and/or anti-aging effects of cosmetic products and/or therapeutic methods considering that many cosmetic products and therapeutic methods are proposed to restore the human skin toward a younger age. The results can also be used for dermatological diagnoses.

As a non-invasive tool, only virtual-level transition is involved in the in vivo skin imaging and thus no energy is left in the human skin. Accordingly, no photodamage is caused to the human skin.

In practice, the observation of the human skin is limited to a certain time, such as up to about 30 minutes, and average power applied to the human skin is controlled within a safety range, such as under about 100 milliwatts (mW). It is to be noted that the time and the power are subject to change according to the tested patients' tolerance and patience. Experimental results show that the in vivo skin imaging is comfortable and does not cause any itch or pain.

In addition, no dye is needed because that the human skin is observed by the HGM device 100, whereby damage to the human skin occurring in conventional skin biopsy methods is also eliminated.

The HGM device 100 includes a laser device 110, a beam shaping device 120, a scanning device 130, an objective lens device 140, a holding device 150, a beam splitting device 160, a filtering device 170, a detecting device 180, and an image processing device 190.

The laser device 110 is configured to emit a laser beam 11b with a predetermined wavelength, pulse width, repetition rate, and output power. The predetermined wavelength falls into a range from about 1200 nanometers (nm) to 1330 nm, such as about 1230 nm, 1250 nm, and 1260 nm. The predetermined pulse width falls into a range from about 15 femtoseconds (fs) to about 500 fs, such as about 51 fs, 100 fs, and 200 fs. The predetermined repetition rate falls into a range from about 10 million hertz (MHz) to about 150 MHz, such as about 50 MHz, 80 MHz, 95 MHz, and 110 MHz. The predetermined output power falls into a range from about 10 milliwatts (mW) to about 1500 mW, such as about 320 mW, 500 mW, 580 mW, and 800 mW.

The laser beam 11b is processed into the excitation light by the beam shaping device 120, the scanning device 130, and the objective lens device 140.

By employing the predetermined wavelength, the pulse width, the repetition rate, and the output power, the section images of the SHG, THG or combination of SHG and THG of the excitation light are clear (i.e., of relatively high resolution and contrast) and distinct, the output power applied to the human skin is controlled to the safe range, and a penetration depth of the excitation light can be greater than about 300 microns (um) or even approach about 1 millimeter (mm).

In this embodiment, the laser device 110 includes a pump source 111, a gain medium 112, an optical cavity 113, and a laser controller 114.

The pump source 111 is configured for supplying energy to the gain medium 112 in a form of electrical current or pumping light having a wavelength different from the predetermined wavelength. The gain medium 112 is configured for amplifying light of the predetermined wavelength. The optical cavity 113 is configured for bouncing the amplified light back and forth, passing through the gain medium 112, such that the amplified light is amplified each time passing the gain medium 112 and comes out as the laser beam 11b. The laser controller 114 is in communication with the pump source 111, the gain medium 112, and the optical cavity 113 and is configured to control various parameters of the laser beam 11b, such as the wavelength, the pulse width, the repetition rate, and the output power.

The pump source 111 can be but is not limited to a semiconductor laser, an ytterbium fiber laser with a wavelength at about 1060 nm, a neodymium doped yttrium aluminum garnet laser, a titanium doped sapphire laser with a wavelength at about 800 nm, or a large-mode-area ytterbium doped photonic crystal fiber (PCF) laser with a wavelength of about 1040 nm, a repetition rate of about 50 MHz, and a pulse width of about 100 fs.

The gain medium 112 can be but is not limited to chromium doped forsterite.

The optical cavity 113 can be but is not limited to an optical parametric oscillator (OPO) or a PCF oscillator. The optical cavity 112 can include optical element of special designs such as curvatures, coatings for obtaining the predetermined parameters of the laser beam 11b such as the predetermined wavelength and pulse width.

In this embodiment, the laser source 11 can be a femtosecond chromium doped forsterite laser with the predetermined wavelength of about 1230 nm, the predetermined pulse width of about 100 fs, the predetermined repetition rate of about 110 MHz, and the predetermined output power of about 500 mW pumped by the about 1060 nm ytterbium fiber laser.

Alternatively, the laser source 11 can be a femtosecond chromium doped forsterite laser with the predetermined wavelength of about 1260 nm, the predetermined pulse width of about 51 fs, the predetermined repetition rate of about 95 MHz, and the predetermined output power of about 800 mW pumped by the about 1060 nm ytterbium fiber laser.

In another embodiment, the laser source 11 can be a femtosecond OPO laser with the predetermined wavelength of about 1250 nm, the predetermined pulse width of 200 fs, the predetermined repetition rate of about 80 MHz, and the predetermined output power of about 580 mW, pumped by the about 800 nm titanium doped sapphire laser.

In further another embodiment, the laser source 11 can be a femtosecond laser with the predetermined wavelength of about 1260 nm, the predetermined repetition rate of about 50 MHz, the predetermined pulse width of about 100 fs, and the predetermined output power of 320 mW based on a 1040 nm, 50 MHz, 100 fs amplified output of the large-mode-area ytterbium-doped PCF oscillator-amplifier laser.

The beam shaping device 120 is positioned in a path of the laser beam 11b and configured for shaping the laser beam 11b into a shaped beam 12b having a shape, size, and collimation degree complying with requirements of the scanning. In this embodiment, the beam shaping device 120 can include collimator lenses for collimating the laser beam 11b.

In other embodiments, if the laser beam 11b already satisfies the requirement of scanning, the beam shaping device 120 can be omitted.

Also referring to FIG. 2, the scanning device 130 and the objective lens device 140 are positioned along a path of the shaped beam 12b and are configured for directing the shaped beam 12b as a scanning beam 14b which is focused into the human skin, e.g., the sample 20, by the objective lens device 140 to form a scanning point (x, y, z). The scanning device 130 and the objective lens 140 are configured for moving the scanning point (x, y, z) to perform a three-dimensional scanning operation on the human skin, e.g., the sample 20. The scanning beam 14b functions as the excitation light.

The scanning device 130 can include a pair of mirrors 131, a scanning motor 132, and a scanning controller 133.

The mirrors 131 are positioned along the path of the shaped beam 12b. The scanning motor 132 is coupled to the mirrors 131. The scanning controller 133 is in communication with the scanning motor 132 and configured for controlling the scanning motors 132 to realize fast and precise positioning of the mirrors 131 for deflection of the shaped beam 12b.

For example, one of the mirrors 131 can move the scanning point (x, y, z) along the X-direction of an XYZ coordinate system, and the other mirror 131 can move the scanning point (x, y, z) along a Y-direction of the XYZ coordinate system, thus realizing an XY-plane scanning operation within one of the stacks of the human skin, e.g., the sample 20. The XYZ coordinate system is established in such a manner that a Z-direction of the XYZ coordinate system is substantially perpendicular to a depth of the human skin. The Z direction movement is accomplished either by the displacement of the objective lens device 140 or by the sample 20 or by both of the mentioned. As such, by scanning each XY-plane, a sectioned image of each stack of the human skin can be formed.

The scanning motor 132 is typically based on moving magnet technology.

The scanning controller 133 is in communication with the scanning motor 132 and is configured for control various parameters of the scanning, such as a scanning range of the XY-plane, step sizes in the X-direction and the Y-direction, and a scanning speed. The scanning range of the XY-plane can be but not limited to about 120 um times about 120 um. That is, each sectioned image has a 120 um*120 um size. The step size in the X-direction and the Y-direction can both be but is not limited to about 120/512 um. That is, each sectioned image has 512*512 pixels. The scanning speed can be but is not limited to about 2.7/262144 second for each step. That is, the sectioned images have a frame speed of about 2.7 seconds.

In this embodiment, the scanning device 130 can have a galvo pair scanning mirrors or galvo-resonance scanning mirrors schema and outputs a rotating beam 13b.

The objective lens device 140 includes a tube lens 141, an objective lens 142, an objective motor 143, and an objective controller 144.

The tube lens 141 and the objective lens 142 are positioned along the path of the rotating beam 13b. The tube lens 141 is configured for diverging the rotating beam 13b to be a light cone 14c, which is focused by the objective lens 142 into the human skin, e.g., the sample 20. That is, the objective lens 142 is configured for focusing the light cone 14c into the human skin, e.g., the sample 20. The objective motor 143 is coupled to the objective lens 142. The objective controller 144 is in communication with the objective motor 143 and configured for controlling the objective motor 143 to move the objective lens 142 along a direction that is substantially parallel with the Z-direction, thus moving the scanning point (x, y, z) along the Z-direction. As such, the sectioned images of different stacks of the human skin at different depths thereof can be obtained by moving the objective lens 142 after each XY-plane scanning operation.

In this embodiment, the objective lens 142 can be but is not limited to an infrared water immersion objective lens.

The objective controller 144 is configured for controlling various parameters of the scanning, such as a step size in the Z-direction and a scanning depth. The step size in the Z-direction can be about 5 um. The scanning depth can be about 300 um.

The holding device 150 is positioned adjacent to the objective lens 142 and configured for holding and stabilizing the human skin, e.g., the sample 20. During the present in vivo skin imaging, vibration of the human skin, e.g., the sample 20, due to, e.g., breathing, can lead to image blurring. As such, the holding device 150 is employed for stabilization of the human skin, e.g., the sample 20

In this embodiment, the holding device 150 includes a substantially disk-like main body 151 and a vacuum source 152. The main body 151 defines a substantially circular observation window 153 through a substantially central portion thereof. The main body 151 also defines a number of vents 154 surrounding the window 153. The objective lens 142 is inserted into the window 153. The vents 154 are communicated with the vacuum source 152.

As such, the human skin, e.g., the sample 20, can be vacuum-held by the vacuum source 152 through the vents 154 and is exposed to the scanning beam 14b from the objective lens 142 via the window 153 after being put on the main body 151.

It could be understood that the holding device 150 is not limited to this embodiment but can take other forms depending on needs. For example, in other embodiments, the human skin, e.g., the sample 20, can be stabilized by magnetic mechanisms or adhesive tapes.

For each scanning point (x, y, z), an observation beam 14e containing SHG 14s and THG 14t of the excitation light is induced and reflected off due to SHG and THG effects.

SHG and THG effects are nonlinear optical processes, in which photons interacting with a nonlinear material are effectively combined to form new photons with twice and triple the energy, and therefore twice and triple a frequency of photons of the excitation light.

SHG often occurs in a noncentrosymmetric and continuous structured media, especially nano-structures, such as stacked membranes, aligned protein structures, and microtubule arrays. However, SHG is not suitable for an observation of interfaces in biological samples.

Since all materials have non-varnishing third order coefficient and since the coefficient is different at different portions of an observed sample, which induces variation of THG intensity, THG is commonly used in non-linear scanning microscopic imaging process. Since THG often occurs in the interface, THG is not suitable for observation of bulk noncentrosymmetric media, which, however, can be clearly inspected by means of SHG.

As such, the SHG and the THG can be combined to observe entire structures of the biological samples.

The beam splitting device 160 is positioned in a path of the observation beam 14e and is configured for separating and directing the SHG 14s and the THG 14t to transmit separately to facilitate independent processing of the SHG 14s and the THG 14t. The beam splitting device 160 includes a first dichromatic beam splitter (DBS) 161 and a second DBS 162.

The first DBS 161 is positioned between the tube lens 141 and the objective lens 142 and configured for passing the light cone 14c directly but deflecting the observation beam 14e. As such, the observation beam 14e is separated from the light cone 14c for further processing.

The second DBS 162 is positioned in the path of the observation beam 14e reflected off from the first DBS 161 and is configured for passing the SHG 14s directly but deflecting the THG 14t. As such, the SHG 14s and the THG 14t are separated and directed to different directions for respective and independent processing.

In this embodiment, the first DBS 161 works at about 850 nm and the second DBS 162 works at about 490 nm and both are but is not limited to about 45-degree tilted in relative with a transmitting direction of the observation beam 14e.

However, configurations of the first DBS 161 and the second DBS 162 are not limited to this embodiment but can be changed depending on needs. For example, the first DBS 161 and the second DBS 162 can work at other wavelengths depending on needs. Further, the second DBS 162 can alternatively pass the THG 14t while reflect the SHG 14s.

The filtering device 170 includes a first filter 171 located in a path of the observation beam 14e and second and third filters 172, 173 positioned in paths of the SHG 14s and the THG 14t, respectively. The second and third filters 172, 173 are configured for filtering noises out of the SHG 14s and the THG 14t to facilitate further processing of the SHG 14s and the THG 14t, e.g., increasing signal-to-noise ratio of the sectioned images.

The first filter 171 is positioned between the first DBS 161 and the second DBS 162 and is configured for filtering noises out of the observation beam 14e consisting of the SHG 14s and THG 14t. The second filter 172 is positioned in the path of the SHG 14s from the second DBS 162 and configured for filtering noises out of the SHG 14s. The third filter 173 is positioned in the path of the THG 14t from the second DBS 162 and is configured for filtering noises out of the THG 14t.

In this embodiment, the first filter 171 works at wavelengths from about 400 nm to about 700 nm (e.g., a color filter), the second filter 172 works at wavelengths from about 600 nm to about 650 nm, and the third filter 173 works at wavelength from about 400 nm to about 430 nm (i.e., both are band-pass filters).

The specific wavelengths given above for the first filters 171, the second filter 172, and the third filter 173 are just for exemplarily illustrate the present disclosure and should not limited the scope of this invention.

Configuration of the filtering device 170 is not limited to this embodiment but can take other forms in other embodiments depending on needs.

The detecting device 180 is positioned in paths of the SHG 14s and the THG 14t and is configured for detecting and converting the filtered SHG 14s and the THG 14t of each scanning point (x, y, z) into electrical signals, such as electric charges, currents, and voltages, of values that are proportional to intensities of the SHG 14s and the THG 14t. The first detector 181 and the second detector 182 also can convert the electrical signals into digital signals by employing analog-to-digital (AD) technologies to facilitate further processing.

The detecting device 180 includes a first detector 181 and a second detector 182. The first detector 181 is positioned in the path of the SHG 14s from the second filter 172 and is configured for detecting the SHG 14s of each scanning point (x, y, z). The second detector 181 is positioned in the path of the THG 14t from the third filter 173 and is configured for detecting the THG 14t of each scanning point (x, y, z).

In this embodiment, the first detector 181 and the second detector 182 are photomultiplier tubes. However, configuration of the detecting device 180 is not limited to this embodiment but can take other forms in other embodiments depending on needs.

Referring to FIG. 1, the image processing device 190 is in communication with the scanning controller 133, the objective controller 144, and the detecting device 180 and configured for synchronizing the detecting device 180 with the scanning controller 133 and the objective controller 144. The image processing device 190 thus is capable of reading the electric signals or digital signals corresponding to the SHG 14s and the THG 14t of each scanning point (x, y, z) and plotting the sectioned images of the SHG 14s, the THG 14t, or combination of the SHG 14s and the THG 14t of each scanning point (x, y, z) with assistance of properties of the SHG 14s and the THG 14t (i.e., the SHG 14s is suitable for observation of bulk noncentrosymmetric media while the THG 14t is suitable for observation of interfaces).

In this embodiment, the sample 20 is the human skin of the forearm of a 24-year female. As shown in FIG. 3, the sectioned images of different stacks including the stratum corneum (SC), the stratum granulosum (SG), the stratum spinosum (SS), the stratum basale (SB), the papillary dermis (PD), and the upper reticular dermis (RD) are illustrated. Depths of the sectioned images are labeled.

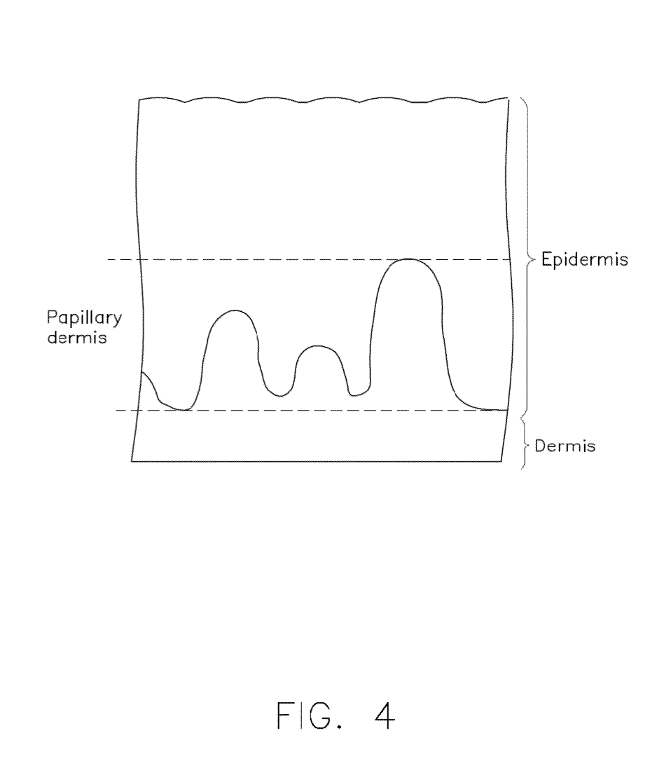

Referring to FIG. 4, briefly, the human skin includes, in this order from a superficial layer to a deep layer of human skin, the epidermis and the dermis. The dermis includes, in this order from the shallow to the deep layers, the papillary dermis and the upper reticular dermis. The papillary dermis includes a number of dermal papillae.

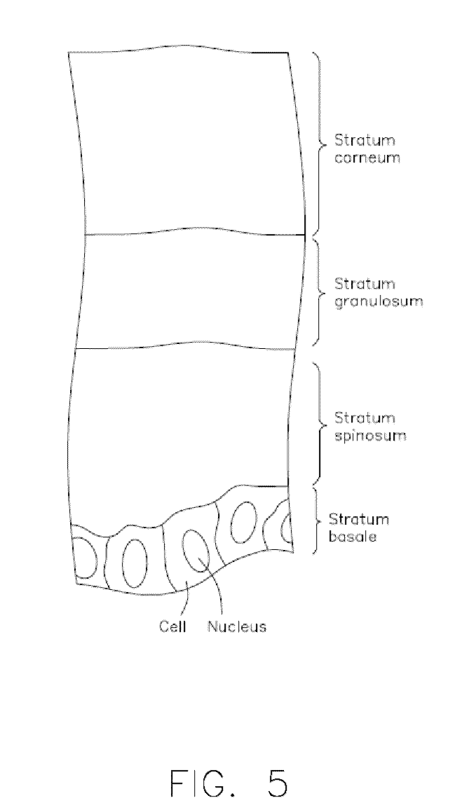

As referring to FIG. 5, in brief, the epidermis includes, form the superficial to the deep layers, the stratum corneum, the stratum granulosum, the stratum spinosum, and the stratum basale. The stratum basale includes a number of substantially honeycomb-like basal cells, each of which includes a nucleus.

To reveal the relationships of the morphological features of the human skins and human chronological ages, a population study is conducted by the system 10 on 48 Asian subjects with 7 females and 8 males aged 19-29 years, 13 females and 6 males aged 30-59 years, and 8 females and 6 males aged 60-79 years, of Fitzpatrick skin phototype III or IV. Each subject is observed by the HGM device 100 to obtain a corresponding group of sectioned images. The study is conducted according to the Declaration of Helsinki Principles, and the protocol is approved by the Institutional Review Board of National Taiwan University Hospital. Informal consent is obtained from each subject prior to the study.

Analyses on various morphological features including the following ones that indicate relationships between the morphological features and the human chronological ages are performed by the analysis device 200 by processing each group of sectioned images.

1. Height and Volume of Papilla

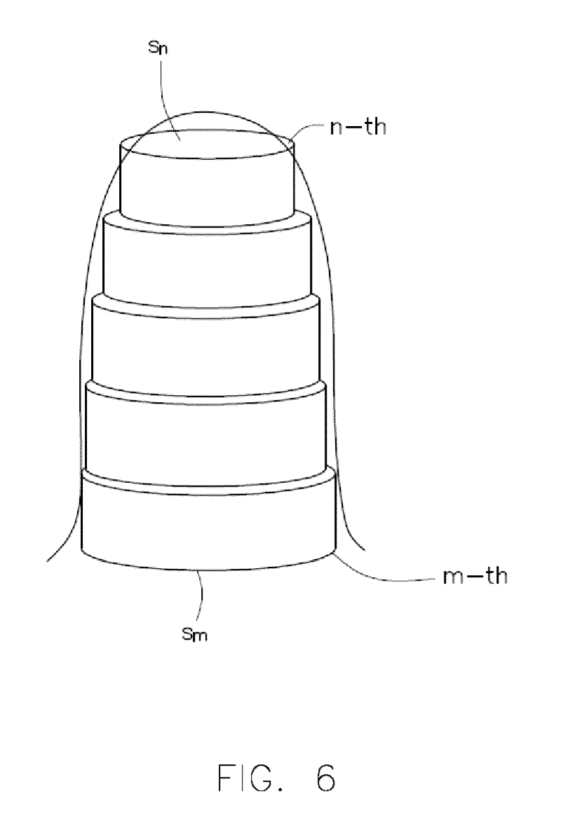

Referring to FIG. 6, for each group of sectioned images of the SHG 14s or combination of the SHG 14s and THG 14t, each papilla is recognized by processing the sectioned images, in this order from the superficial to the deep layers, and the n-th sectioned image in which the papilla is first present and the m-th sectioned image in which the papilla is first vanished are found by the analysis device 200, wherein n, m are positive integers and m>n. Then, a height of the papilla (i.e., isolated dermal papilla height) can be calculated by the formula: H=z*|(m−n) using the analysis device 200, wherein H is the papilla height and z is the step size of the scanning in the Z-direction.

In addition, an area of the papilla in each sectioned image in which the papilla is present is measured by the analysis device 200. Then, a volume of the papilla (i.e., isolated dermal papilla volume) can be calculated by the formula:

$$V = {\sum }_{i = m}^{i = n} z * {s}_{i}$$

using the analysis device 200, wherein V is the volume and sᵢ is the area of the papilla in the i-th sectioned image, and n≦i≦m.

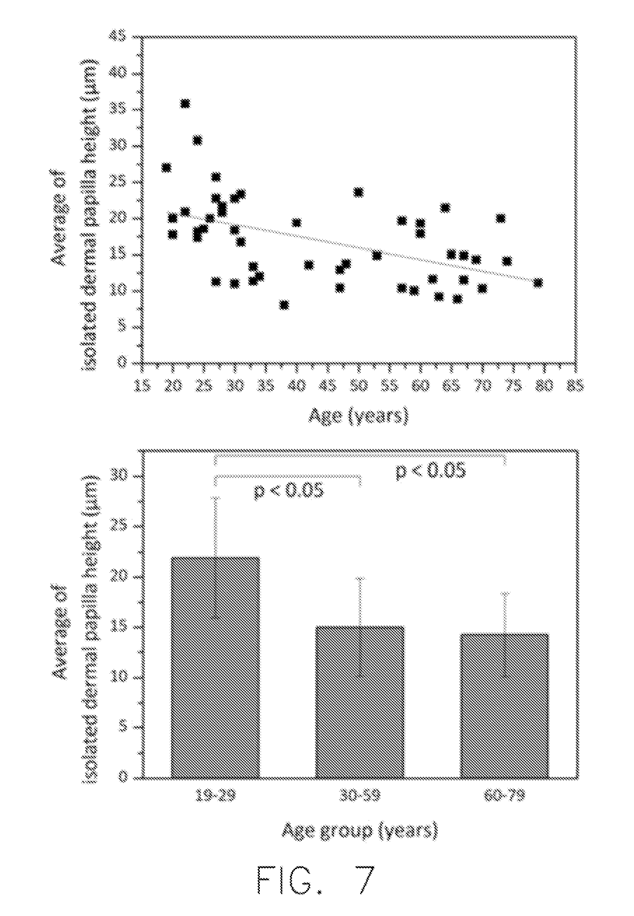

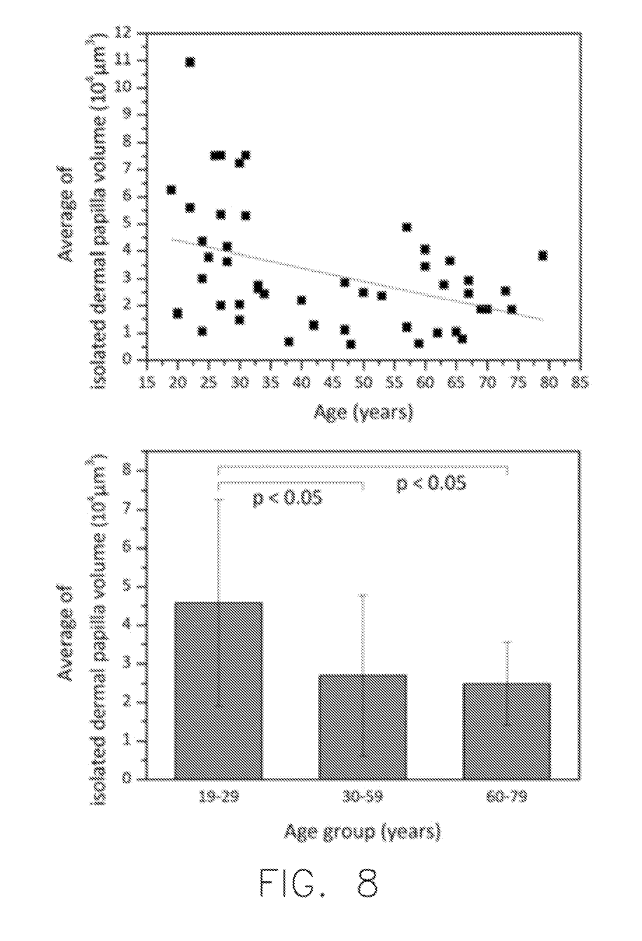

Referring to FIGS. 7-8, statistical results show that the average height and average volume of the papillae of the skin decrease with aging.

2. Thickness of Epidermis Including Papillae

Referring back to FIG. 5, for each group of sectioned images, the M-th sectioned image in which all the papillae are first vanished is found by the analysis device 200. A thickness of the epidermis including the papillae, i.e., viable epidermis (including the rete ridge), is calculated by the formula: T=M*z using the analysis device 200, wherein T is the thickness of the epidermis including the papillae and M is a positive integer.

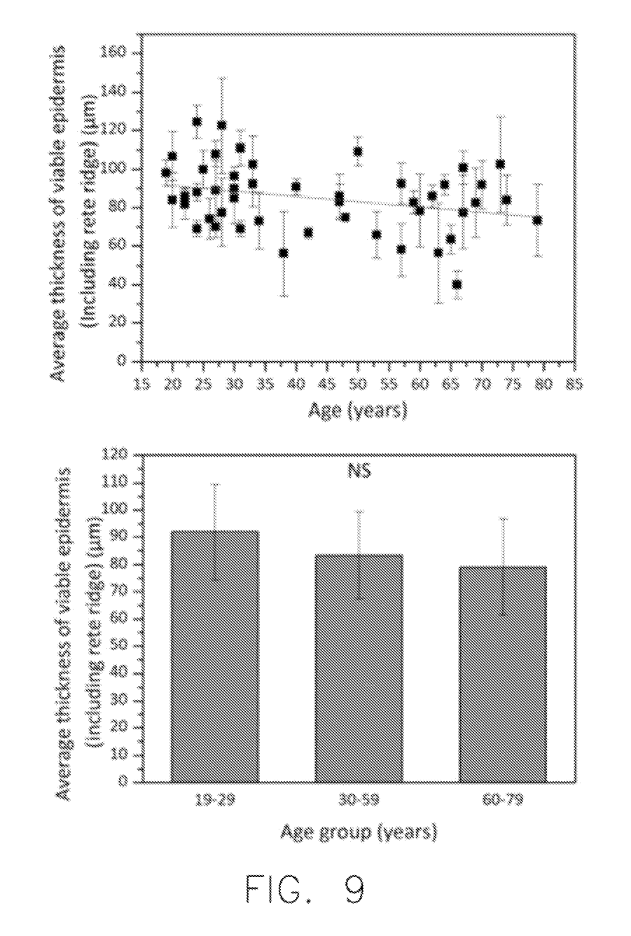

Referring to FIG. 9, statistical results show that the average thickness of the viable epidermis including the papillae of the skin decreases with aging.

3. Flatten of Epidermis-Dermis Junction

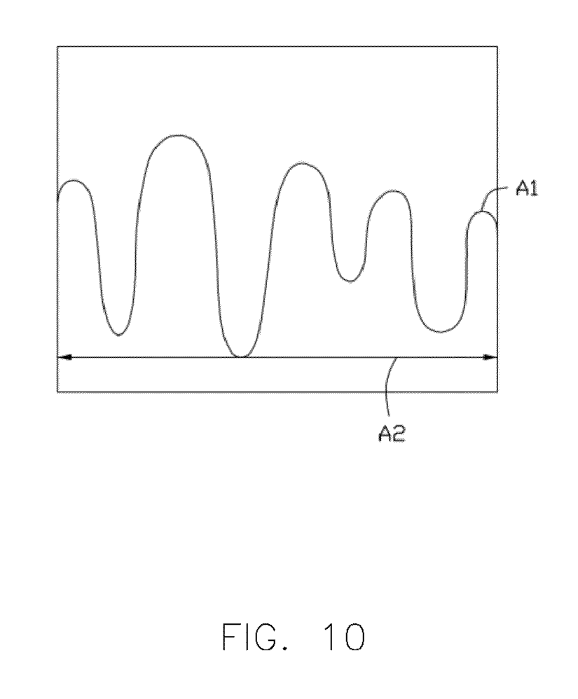

Referring to FIG. 10, for each group of sectioned images of the SHG 14s or combination of the SHG 14s and the THG 14t, the N-th sectioned image in which any papilla is first present is found by the analysis device 200. Then, an interface area is calculated by the formula:

$$A 1 = {\sum }_{i = M}^{i = N} z * {L}_{i}$$

using the analysis device 200, wherein Lᵢ is a circumference length of the papillae in the i-th sectioned image. In addition, an area of the papillae projecting onto the M-th sectioned image is measured by the analysis device 200. Next, a 3D interdigitation index is calculated by the formula:

$$I = \frac{A 1}{A 2}$$

using the analysis device 200, wherein A2 is the area of the papillae projecting onto the M-th sectioned image.

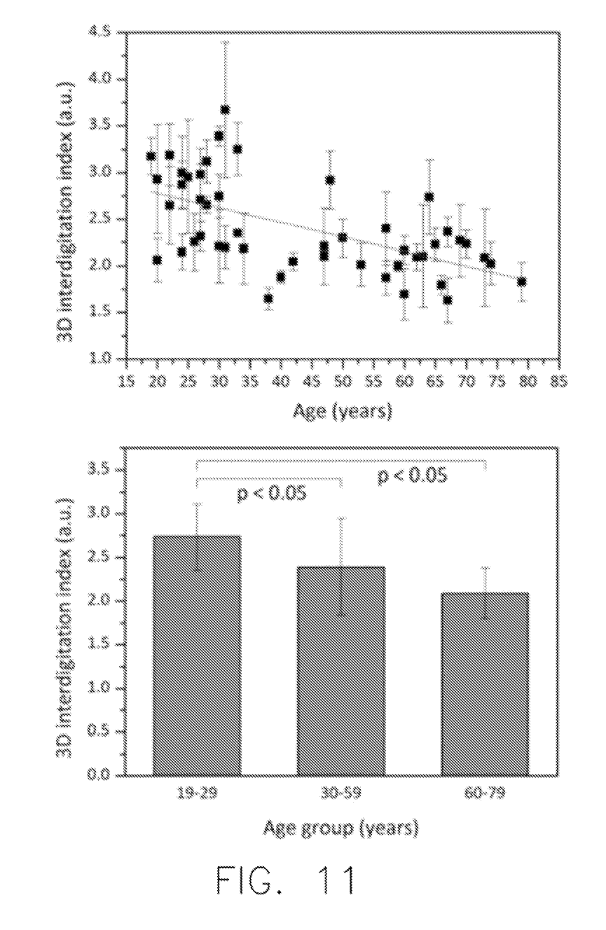

Referring to FIG. 11, statistical results show that the 3D interdigitation index decreases with aging.

4. Cellular and Nucleus Areas of Stratum Basale

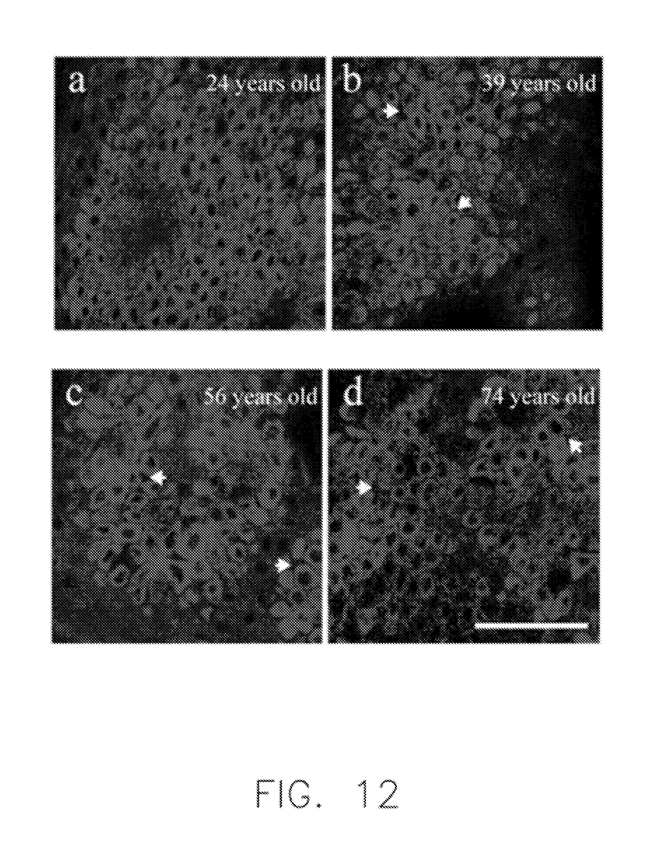

Referring to FIG. 12, for each group of sectioned images of the THG 14t or combination of the SHG 14s and the THG 14t, cells and nuclei of the stratum basale are recognized and cellular and nucleus areas of the stratum basale are measured by the analysis device 200.

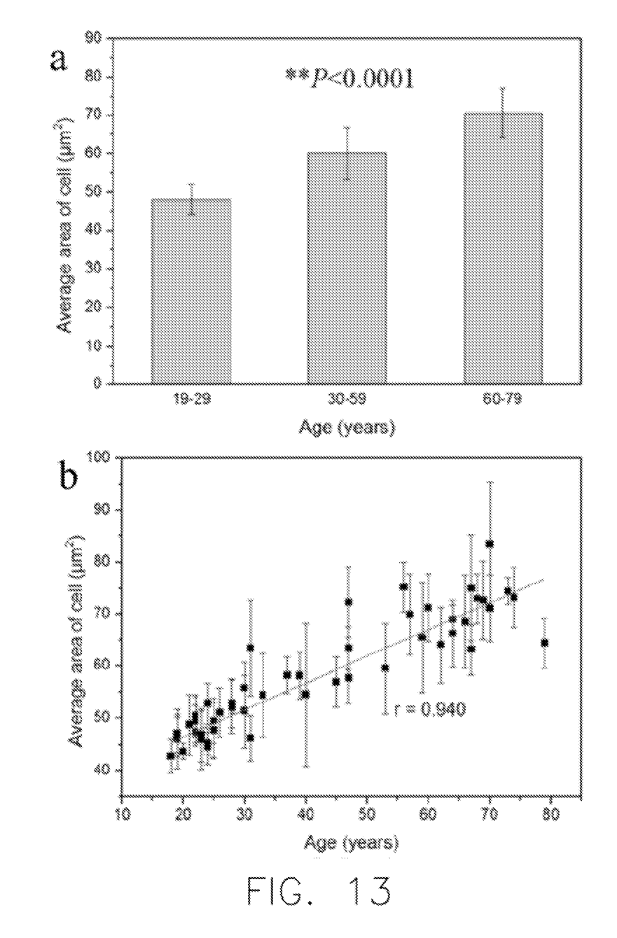

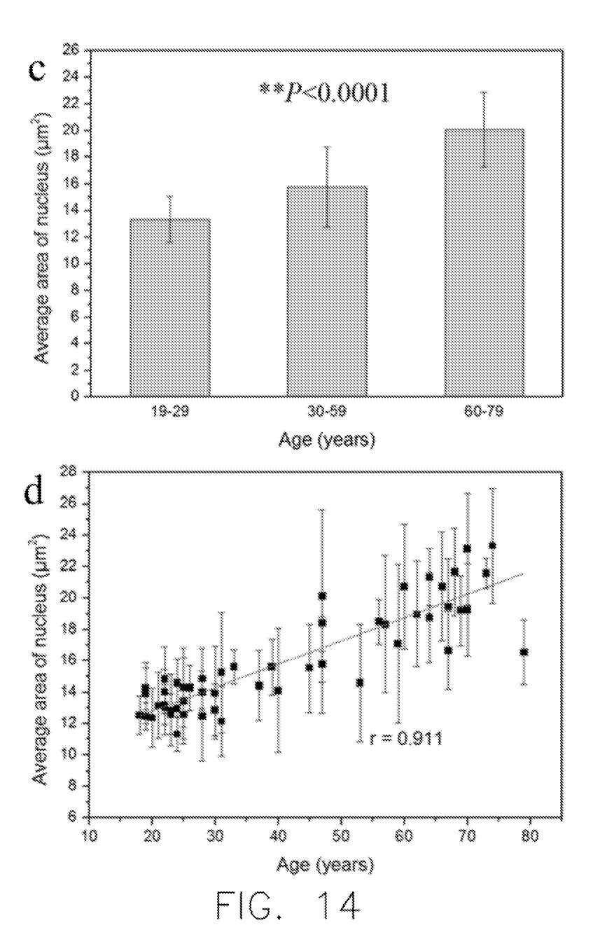

Referring to FIGS. 13-14, statistical results show that both the average cellular and nucleus areas of the skin increase with aging.

Various components of the image processing device 190 and the analysis device 200 can be integrated into a single control unit, or alternatively, can be implemented in software algorithm or hardware, such as, for example, a field programmable gate array (FPGA) or an application specific integrated circuit (ASIC). In addition, image processing and analysis method steps may be performed by the same control unit or a processor executing instructions organized into a program module or a custom designed state machine. As such, it is intended that the processes described herein be broadly interpreted as being equivalently performed by software, hardware, or a combination thereof. As previously discussed, software modules can be written, via a variety of software languages, including C, C++, Java, Visual Basic, and many others. These software modules may include data and instructions which can also be stored on one or more machine-readable storage media, such as dynamic or static random access memories (DRAMs or SRAMs), erasable and programmable read-only memories (EPROMs), electrically erasable and programmable read-only memories (EEPROMs) and flash memories; magnetic disks such as fixed, floppy and removable disks; other magnetic media including tape; and optical media such as compact discs (CDs) or digital video discs (DVDs). Instructions of the software routines or modules may also be loaded or transported into the wireless cards or any computing devices on the wireless network in one of many different ways. For example, code segments including instructions stored on floppy discs, CD or DVD media, a hard disk, or transported through a network interface card, modem, or other interface device may be loaded into the system and executed as corresponding software routines or modules. In the loading or transport process, data signals that are embodied as carrier waves (transmitted over telephone lines, network lines, wireless links, cables, and the like) may communicate the code segments, including instructions, to the network node or element. Such carrier waves may be in the form of electrical, optical, acoustical, electromagnetic, or other types of signals.

In operation, the system 10 is first provided. Then the population study can be performed to establish the database. Finally, the sample 20 can be observed to obtain the corresponding group of section images. Then the analyses can be performed on the sectioned images of the sample 20 to obtain the morphological features of the sample 20 to assist to estimate a chronological age of the person.

As mentioned above, the results of in vivo skin imaging can also be used for dermatological diagnoses. In this regard, several examples are provided below to further illustrate characteristics of diagnostic methods of the present disclosure. It should be apparent that the skin imaging results can be used for examining any morphological feature of skin tissue of subjects, or patients, without being limited to those features exemplified below for diagnosing skin diseases. As used herein, the term “skin disease” means any undesirable skin condition or symptom suffered by patients. The term “morphological feature” as used herein refers to, but not limited to, distribution, size, shape, and proliferation of cells, size and shape of nuclei of the cells, and distribution and shape of collagen fibers.

In the following examples, the HGM device 100 is applied to patients suffered from skin diseases to characterize distinct criteria showing on their skin tissues in contrast to that showing on healthy skin. The operational procedures, conditions and configurations of the HGM device 100 are identical to those mentioned above. In one embodiment, section images of skin tissue of patients may be formed through signals of SHG, THG, or combination thereof from an excitation light used by the HGM device 100 to in vivo scan the skin tissue, in which the excitation light has a wavelength in a range from about 1200 nm to 1330 nm. In the following examples, a laser beam having a wavelength of 1230 nm is used to in vivo scan the skin tissue of patients, by which the low illumination attenuation can be achieved, so as to allow SHG/THG to fall within the visible spectrum.

### Example 1

### Diagnosis of Melanoma

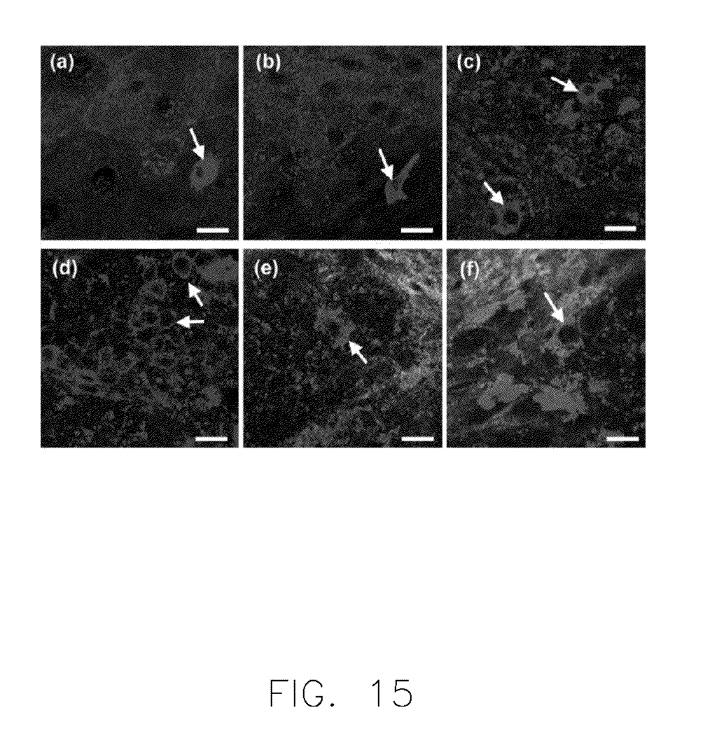

Melanoma is the most serious type of skin cancer, which is caused by malignant changes in melanocytes. Sectioned images of the skin tissue of a patient are obtained by the HGM device 100, and taken at different depths relative to the skin surface (10, 40, 65, 80, 110, and 150 μm) (see FIG. 15). Morphological features, including distribution of melanocytes, size of melanocytes, shape of melanocytes, size of melanocytic nuclei, shape of melanocytic nuclei, intracellular distance of melanocytes, and proliferation of melanocytes, are identified and analyzed through processing the sectioned image. Irregularly shaped cells with strong THG signals are found in the stratum granulosum, referring to arrows in FIGS. 15(a)-(b). The distribution of these cells is similar to the pagetoid spread of melanocytes observed in the conventional pathologic sections of melanoma. Deeper into the skin lesion, the proliferation of polymorphous THG-bright cells in the epidermis (see arrows in FIGS. 15(c)-(e)) and in the dermis (see arrow in FIG. 15(f)) is evident. These melanocytes with bright cytoplasm and dark nuclei are more polymorphic and larger (about 25 μm in diameter) than those seen in BCC (about 15 μm in diameter). In addition, the large intercellular distance is revealed in the tumor. Due to these unique features, it can be determined that the patient suffers from melanoma. Among these features analyzed, proliferation of polymorphous melanocytes, large intercellular distance, and pagetoid spread of melanocytes are crucial criteria to determine whether a patient has melanoma.

### Example 2

### Diagnosis of Basal Cell Carcinoma

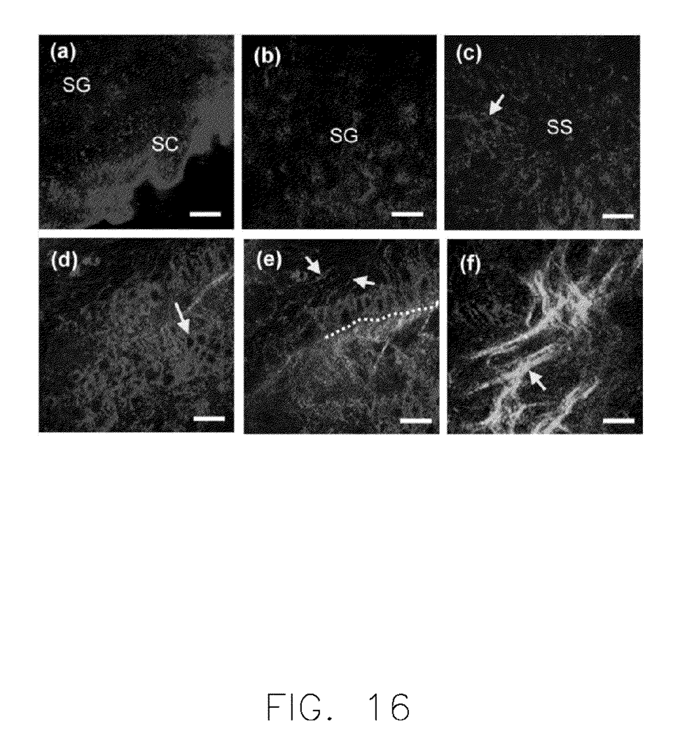

Basal cell carcinoma (BCC) is the most common type (>80%) of skin cancer, which grows from the basal cell layer. Sectioned images of the skin tissue of a patient are obtained by the HGM device 100, and taken at different depths relative to the skin surface (5, 15, 25, 45, 65, and 85 μm) (see FIG. 16). In FIGS. 16(a)-(c), “SC” stands for stratum corneum; “SG” stands for stratum granulosum; and “SS” stands for stratum spinosum. Morphological features, including shape of basal cells, size of basal cells, distribution of peripheral palisading cells, collagen bundles, dendritic processes of cells, and proliferation of basal cells, are identified and analyzed through processing the sectioned image. It is found that THG-bright cells rising from the stratum basale and extending into the dermis appear polymorphous with variations in size and shape (see FIGS. 16(d)-(f)). Moreover, the presence of elongated, peripheral palisading cells in the tumor nodules is detected (see dashed line in FIG. 16(e)). Collagen bundles surrounding the tumor nests become more thickened and coarser than their normal counterparts, as revealed from SHG images (see arrow in FIG. 16(f)). The presence of a large number of cells with dendritic processes is also found (see arrows in FIGS. 16(c) and (e)). Using these features, it can be determined that the patient has BCC. Among these features analyzed, peripheral palisading cells, proliferation of polymorphous basaloid cells, elongated cells/nuclei and collagen changes are crucial criteria to determine whether a patient has BCC.

### Example 3

### Diagnosis of Melanocytic Nevus

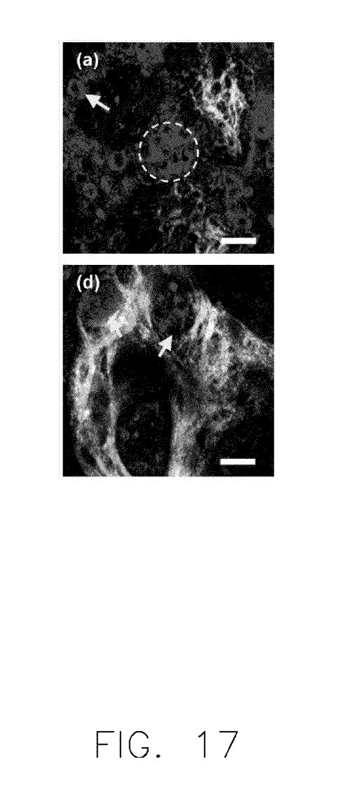

Melanocytic nevus is a form of benign neoplasm that can be divided into three nevus types according to the locations of the nevus cells, which are derived from melanocyte. Sectioned images of the skin tissue of a patient suffered from melanocytic nevus are obtained by the HGM device 100 (see FIG. 17). Morphological features, including size of nevus cells, shape of nevus cells, and distribution of nevus cells, are identified and analyzed through processing the sectioned image. The presence of the aggregation of THG-bright cells in the dermo-epidermal junction (see dashed circle in FIG. 17(a)) and in the dermis (see arrow in FIG. 17(d)) are observed in junctional and intradermal melanocytic nevi, respectively. The nevus cells in both the dermo-epidermal junction and dermis from the HGM images of compound nevi are also observed. The cells in the nests, which are monomorphic in size and shape, are observed using THG imaging. The scattered melanocytes (see arrow in FIG. 17(a)), which are larger than basal cells, are also found in the stratum basale. In addition, the THG-bright cells, distributing linearly along the elongated rete ridges, represent basal hyperpigmentation in the junctional nevus. Using these features, the three kinds of melanocytic nevus can be determined. Among these features analyzed, monomorphous cell nests, normal epithelial stratification, and elongation of rete ridges are crucial criteria to determine whether a patient has melanocytic nevus.

### Example 4

### Diagnosis of Seborrheic Keratosis

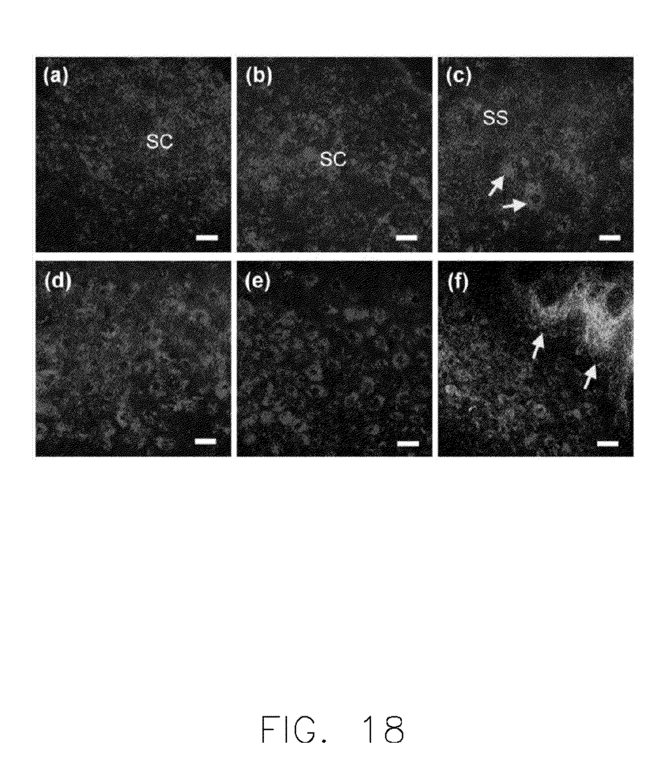

Seborrheic keratosis (SK) is the most common benign tumor in older individuals. It originates from the growth of keratinocytes, and involves only the epidermis. Sectioned images of the skin tissue of a patient are obtained by the HGM device 100 and taken at different depths relative to the skin surface (5, 40, 65, 85, 105, and 130 μm) (see FIG. 18). Morphological features, including proliferation of keratinocyte, keratin pearl, size of monomorphous keratinocytes, shape of monomorphous keratinocytes, are identified and analyzed through processing the sectioned image. It is found that the proliferation of THG-bright keratinocytes occurred in the lower epidermis. Due to the increased melanin content, THG-bright cytoplasm is observed in some spinous cells and in the majority of monomorphous basaloid cells (see arrows in FIG. 18(c)). Compared with BCC and melanoma, SK exhibits linearly aligned normal basal cells in the intact dermo-epidermal junction (see arrows in FIG. 18 (f)) in the HGM imaging. Due to these unique features, it can be determined that the patient suffers from SK. Among these features analyzed, proliferation of monomorphous basaloid cells and acanthotic epidermis are crucial criteria to determine whether a patient has SK.

### Example 5

### Diagnosis of Actinic Keratosis

In another example, the HGM device 100 is used to in vivo scan the skin tissue of a patient to determine whether the patient suffers from actinic keratosis (AK). Sectioned images are taken at different depths relative to the skin surface (0, 20, 50, 70, and 100 μm). Morphological features, including distribution of keratinocytes, distribution of collagen, shape of basal cells, size of basal cells, and size of nuclei with prominent nucleoli, are identified and analyzed through processing the sectioned image. Atypical keratinocytes occupy the lower aspects of the epidermis. The basal cells are found to be irregular and enlarged. Enlarged nuclei with prominent nucleoli are observed. Due to these unique features, it can be determined that the patient suffers from AK.

### Example 6

### Diagnosis of Extramammary Paget's Disease (EMPD)

In further another example, the HGM device 100 is used to in vivo scan the skin tissue of a patient to determine whether the patient suffers from extramammary Paget's disease (EMPD). Sectioned images are taken. Morphological features, including distribution of Paget's cells, size of Paget's cells, shape of Paget's cells, and distribution of signet ring cells, are identified and analyzed through processing the sectioned image. It is found that Paget's cells are large with amphophilic, granular cytoplasm and prominent atypical nucleus. Some Paget's cells infiltrate into the upper portion of the epidermis. Due to these unique features, it can be determined that the patient suffers from EMPD.

### Example 7

### Diagnosis of Squamous Cell Carcinoma (SCC)

In yet another example, the HGM device 100 is used to in vivo scan the skin tissue of a patient to determine whether the patient suffers from squamous cell carcinoma (SCC). Sectioned images are taken. Morphological features, including size of cell nuclei, shape of cell nuclei, and distribution of cells, are identified and analyzed through processing the sectioned image. Various sizes and shapes of cell nuclei are observed. High nuclear-to-cytoplasmic ratio is also noticed. It is also found that cellular density is high, and tumor cell invades into the underlying dermis. Due to these unique features, it can be determined that the patient suffers from SCC.

The above results suggest that the locations of the cell nests and the morphology of the proliferating THG-bright cells are important for identifying a specific disease. The diagnostic performance of the present disclosure is examined through direct general assessment (overall evaluation) or the use of two imaging criteria (major criteria evaluation). At least two axial sections in the HGM images of each lesion are observed to evaluate the diagnostic performance. In this analysis, sensitivity indicates the percentage of sick people who are correctly identified as having the condition, whereas specificity indicates the percentage of healthy people who are correctly identified as not having the condition. For example, 44 lesions (10 SK and 34 other diseases) are analyzed in SK, and we observe the acanthotic epidermis of 11 lesions. The histological reports reveal that the 11 lesions include 9 SK and 2 other diseases. Therefore, sensitivity of 90% [9/10=90%] and specificity of 94% [(34-2)/34=94%] are obtained. In one example, sensitivity of 92% and specificity of 97% are obtained by the diagnostic method of the present disclosure. In another example, sensitivity of 95% and specificity of 100% are achieved by the diagnostic method of the present disclosure. Good diagnostic performance with high sensitivity and specificity is achieved by the present method. These results indicate that HGM in vivo imaging of the present disclosure can clearly distinguish the specific morphological features and provide accurate and immediate diagnosis.

In one embodiment, the diagnostic method of the present disclosure may be used to differentiate melanoma from BCC, nevi, and SK. In one example, the sensitivity and specificity for differentiation achieved are 100% and 99%, respectively. HGM in vivo imaging of the present disclosure can achieve the excellent capability to clearly distinguish between the epidermis and dermis. This capability is crucial in determining the degree of invasion.

Due to the nonlinearity and virtual-level transition nature in harmonic generation process and the Cr:forsterite excitation laser with a wavelength capable of minimized damage in bio-tissues, the HGM in vivo imaging of the present disclosure possesses great subcellular resolution, adequate penetration depth, while leaving no photodamage to patient's skin in clinical operations.

While there have been illustrated and described what are considered to be example embodiments of the present disclosure, it will be understood by those skilled in the art and as technology develops that various changes and modifications, may be made, and equivalents may be substituted for elements thereof without departing from the true scope of the present disclosure. Many modifications, permutations, additions and sub-combinations may be made to adapt the teachings of the present disclosure to a particular situation without departing from the scope thereof. Accordingly, it is intended, therefore, that the present disclosure not be limited to the various example embodiments disclosed, but that the present disclosure includes all embodiments falling within the scope of the appended claims.

What is claimed is:

1. A method for examining skin tissue of a subject based on in vivo skin imaging, the method comprising:
providing a harmonic generation microscopy (HGM) device;
obtaining at least one sectioned image of the skin tissue of the subject by using the HGM device, wherein the at least one sectioned image is formed through signals of second harmonic generation (SHG), third harmonic generation (THG), or combination of the SHG and THG from an excitation light having a wavelength in a range from about 1200 nm to 1330 nm and being used by the HGM device to in vivo scan the skin tissue; and
identifying and analyzing at least one morphological feature of the skin tissue by processing the at least one sectioned image.

2. The method of claim 1, wherein the excitation light has a wavelength of 1230 nm.

3. The method of claim 1, further comprising determining whether the at least one morphological feature of the skin tissue is related to a skin disease.

4. The method of claim 3, wherein the skin disease is selected from the group consisting of melanoma, basal cell carcinoma, melanocytic nevus, seborrheic keratosis (SK), actinic keratosis (AK), extramammary Paget's disease (EMPD), and squamous cell carcinoma (SCC).

5. The method of claim 1, wherein the at least one morphological feature is selected from the group consisting of distribution, size, shape, and proliferation of cells, size and shape of nuclei of the cells, distribution and shape of collagen fibers, and any combination thereof.

6. The method of claim 5, wherein the cells are selected from the group consisting of melanocytes, basal cells, peripheral palisading cells, keratinocytes, Paget's cells, signet ring cells, and any combination thereof.

7. A method for diagnosing a skin disease in a subject based on in vivo skin imaging, the method comprising:
using a HGM device to in vivo scan a laser beam having a wavelength in a range from about 1200 nm to 1330 nm to a skin tissue of the subject to generate signals of SHG, THG or combination thereof;
converting the signals to at least one sectioned image of the skin tissue;
identifying and analyzing at least one morphological feature of the skin tissue by processing the at least one sectioned image; and
determining whether the at least one morphological feature is related to the skin disease.

8. The method of claim 7, wherein the laser beam has a wavelength of 1230 nm.

9. The method of claim 8, wherein the laser beam is generated by a 1230 nm femtosecond Cr-F laser.

10. The method of claim 7, wherein the skin disease is selected from the group consisting of melanoma, basal cell carcinoma, melanocytic nevus, seborrheic keratosis (SK), actinic keratosis (AK), extramammary Paget's disease (EMPD), and squamous cell carcinoma (SCC).

11. The method of claim 7, wherein the at least one morphological feature is selected from the group consisting of distribution, size, shape, and proliferation of cells, size and shape of nuclei of the cells, distribution and shape of collagen fibers, and any combination thereof.

12. The method of claim 11, wherein the cells are selected from the group consisting of melanocytes, basal cells, peripheral palisading cells, keratinocytes, Paget's cells, signet ring cells, and any combination thereof.

13. The method of claim 7, wherein the skin disease is melanoma.

14. The method of claim 13, wherein the at least one morphological feature is selected from the group consisting of distribution of melanocytes, size of melanocytes, shape of melanocytes, size of melanocytic nuclei, shape of melanocytic nuclei, intracellular distance of melanocyte, proliferation of melanocyte, and any combination thereof.

15. The method of claim 7, wherein the skin disease is basal cell carcinoma.

16. The method of claim 15, wherein the at least one morphological feature is selected from the group consisting of shape of basal cells, size of basal cells, distribution of peripheral palisading cells, collagen bundles, dendritic processes of cells, proliferation of basal cell, and any combination thereof.

17. The method of claim 7, wherein the skin disease is melanocytic nevus.

18. The method of claim 17, wherein the at least one morphological feature is selected from the group consisting of size of nevus cells, shape of nevus cells, distribution of nevus cells, and any combination thereof.

19. The method of claim 7, wherein the skin disease is seborrheic keratosis.

20. The method of claim 19, wherein the at least one morphological feature is selected from the group consisting of proliferation of keratinocyte, keratin pearl, size of monomorphous keratinocytes, shape of monomorphous keratinocytes, and any combination thereof.

21. The method of claim 7, wherein the skin disease is actinic keratosis.

22. The method of claim 21, wherein the at least one morphological feature is selected from the group consisting of distribution of keratinocytes, distribution of collagen, shape of basal cells, size of basal cells, size of nuclei with prominent nucleoli, and any combination thereof.

23. The method of claim 7, wherein the skin disease is extramammary Paget's disease.

24. The method of claim 23, wherein the at least one morphological feature is selected from the group consisting of distribution of Paget's cells, size of Paget's cells, shape of Paget's cells, distribution of signet ring cells, and any combination thereof.

25. The method of claim 7, wherein the skin disease is squamous cell carcinoma.

26. The method of claim 25, wherein the at least one morphological feature is selected from the group consisting of size of cell nuclei, shape of cell nuclei, distribution of cells, and any combination thereof.
